(19) United States
(12) Patent Application Publication
(10) Pub. No.: US 2006/0024705 A1
(43) Pub. Date: Feb. 2, 2006

(54) Molecular analysis of hair follicles for disease

(75) Inventors: Michael Benjamin Centola, Oklahoma City, OK (US); Theodore B. Thederahn, Los Angeles, CA (US); Mark Barton Frank, Edmond, OK (US); Richard Craig Cadwell, Oklahoma City, OK (US); Igor Dozmorov, Oklahoma City, OK (US); Cherie Chappell, Annapolis, MD (US)

Correspondence Address: FULBRIGHT & JAWORSKI L.L.P., 600 CONGRESS AVE., SUITE 2400, AUSTIN, TX, 78701, US

(73) Assignees: Wella AG, Board of Regents of the University of Oklahoma and; Oklahoma Medical Research Foundation

(21) Appl. No.: 11/146,312

(22) Filed: Jun. 6, 2005

Related U.S. Application Data
(60) Provisional application No. 60/577,729, filed on Jun. 7, 2004.

(51) Int. Cl. C12Q 1/68

(52) U.S. Cl. 435/6

(57) ABSTRACT

Methods are provided for the analysis of gene expression utilizing RNA from hair follicles. Methods are also provided for evaluation of the biological activity of a candidate substance, genetic diagnosis, and evaluation of disease, each involving analysis of gene expression utilizing RNA from hair follicles.

This application claims the benefit of U.S. Provisional Application Ser. No. 60/577,729 filed Jun. 7, 2004, the entire disclosure of which is specifically incorporated herein by reference.

### BACKGROUND OF THE INVENTION

1. Field of the Invention

The present invention relates generally to the field of molecular biology. More particularly, it concerns methods for gene expression analysis of hair follicles.

2. Description of Related Art

Gene expression analysis is used to diagnose disease states, predict disease outcomes, monitor and predict responses to therapies, identify novel therapeutic targets, and determine the efficacy of therapeutic agents in vivo. Current methods of obtaining tissue for gene expression analysis are invasive, and include venipuncture and surgical biopsies. Risks to subjects undergoing such procedures include trauma, hematomas, infection, and death. Additionally, such methods require skilled, medically-trained personnel, adding expense.

Obtaining intact RNA is required for gene expression profiling. Currently, it is beyond the capabilities of most clinical facilities to both isolate tissues and produce stable RNA from these tissues. Although whole blood can be lysed on site and the RNA could be stabilized, the majority of the cells in this tissue (i.e., red blood cells) lack nuclei and thus do not produce RNA in response to environmental stimuli, and subsets of nucleated white blood cells with distinct functions are difficult to differentially isolate.

Dissected skin from mice (Schlake and Boehm, 2001) and biopsies from humans (Carroll et al., 2002) have been previously used for microarray gene expression analysis. However, methods such as these (i.e., requiring significant amounts of removed skin) are not viable as non-invasive methods for analysis of gene expression in human subjects. Thus, a need exists for a relatively non-invasive method of gene analysis.

### SUMMARY OF THE INVENTION

The present invention overcomes deficiencies in the prior art by providing methods for gene analysis using tissue from hair follicles. The development of methods in the present invention to isolate and perform gene expression analyses using RNA collected from hair follicles obtained from plucked hairs provides a novel non-invasive method for screening of drugs, diagnosis disease, and various other embodiments.

A first embodiment of the present invention involves a method for determining gene expression comprising the steps of (a) obtaining a hair follicle, (b) isolating RNA from the hair follicle; and (c) detecting the RNA by performing gene expression analysis. In certain embodiments of the present invention, the hair follicle may be obtained from a human. The RNA may be stabilized, amplified, and/or reverse transcribed into DNA. In certain embodiments of the present invention, the gene expression analysis may be microarray analysis. In other embodiments of the present invention, the gene expression analysis is Northern blot analysis, Southern blot analysis, or differential display. The hair follicle may be obtained by plucking a hair.

In certain embodiments of the present invention, the gene expression analysis may be used for disease diagnosis, for predicting outcomes for the treatment of a disease, or for monitoring a response to a stimuli. The disease may comprises a disease selected from the group consisting of cancer, a genetic disease, a viral disease, a fungal disease, a bacterial disease, a cardiac disease, diabetes, a neurodegenerative disease, or an autoimmune disease. In certain embodiments of the present invention, the stimuli is a pharmaceutical or a cosmeceutical. In other embodiments of the present invention, the stimuli is an environmental stimuli. The environmental stimuli may be pollution or a toxin.

The present invention also provides a method for evaluating the biological activity of a candidate substance comprising the steps of exposing a subject to the absence or presence of the candidate substance, obtaining a hair follicle from the subject, isolating RNA from the hair follicle; and detecting the RNA by performing gene expression analysis, wherein a difference in the gene expression analysis is observed as a result of the exposure to the candidate substance as compared to the absence of the exposure to the candidate substance. The RNA may be stabilized. The RNA may be amplified. The RNA may be reverse transcribed into DNA. In certain embodiments of the present invention, the gene expression analysis may be microarray analysis. In other embodiments of the present invention, the gene expression analysis may be Northern blot analysis, Southern blot analysis, or differential display. The subject may be a human. In certain embodiments of the present invention, the candidate substance can affect hair development. Alternatively, the candidate substance may be used to treat a disease selected from the group consisting of cancer, a genetic disease, a viral disease, a fungal disease, a bacterial disease, a cardiac disease, diabetes, a neurodegenerative disease, or an autoimmune disease.

The present invention also provides a method for using gene expression analysis to detect quantative levels of fibroblast growth factor 19 (FGF-19). FGF-19 may be used as a surrogate biomarker for hair loss or hair growth. Hair loss may comprise baldness, pattern baldness, alopecia, thinning hair, or hair loss due to a pharmaceutical, chemotherapeutic or environmental stimuli. Hair growth may be stimulated by treatment of the follicle with FGF-19, FGFR4, or an FGF-19 or FGFR4 agonist or similar pharmaceutical, chemotherapeutic, or cosmeceutical, causing the activation of the FGF19 gene expression pathway, thereby upregulating FGF-19.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Quantitative PCR (QPCR) of RNA from pulled hair samples. QPCR was performed on cDNA derived from pulled hair total RNA preparations and on 10-fold serial dilutions of a commercially available control RNA sample (Universal RNA, Stratagene). The 18S ribosomal housekeeping gene was amplified using Taqman primers and probes. Realtime applification curves are shown on a plot of arbitrary fluorescent units versus cycle time. The amounts of 18S RNA are estimated by placing a threshold value (black line) through the linear portion of the amplification curves. The number of cycles that were required for a given sample to reach the threshold value, denoted Ct or cycle time, are shown in the sample key. Two-fold increases occur in each cycle, therefore differences in Ct among samples are equal to 2^{(Ct)} differences in 18S concentration. Approximately 100 ng of RNA/follicle was obtained in the pulled hair samples as estimated by comparing Ct values of pulled hair RNA to known concentrations of control RNA.

FIG. 2—Variation in Microarray Results Using RNeasy-Purified RNA. Coefficients of variation (CV) for gene expression of each group are shown. Microarray gene expression analysis showed that consistent results can be obtained from a single individual.

FIG. 3—Variation in Microarray Results as a Function of Race. Coefficients of variation (CV) for gene expression of each group are shown. The mean CV is greater for a group of individuals than for samples obtained from the same individual at different times. The mean CV within each race was low, and the distribution of CVs was narrow. The mean and distribution of CVs between different races was higher than that detected within each race. These increased CVs may reflect biological differences in hair follicle gene expression between races.

### DESCRIPTION OF ILLUSTRATIVE EMBODIMENTS

The present invention overcomes deficiencies in the prior art by providing methods for non-invasive gene expression analysis using RNA obtained from hair follicles. The inventors have discovered, surprisingly, that sufficient biological material can be obtained from hair follicles to perform various analyses, and that the RNA thus obtained can be used to quantitate differences in gene expression as compared to various “normal” or “control” samples. The ability to use non-invasive techniques for such assays is a significant improvement over prior approaches.

The present invention finds particular use in the diagnosis of disease states, for the identification of genetic factors governing developmental programs, including defects there, for the identification of drug targets, for development of pharmaceuticals and cosmeceuticals, and in assessing the impact of environmental factors such as toxins and pollution on a subject.

A. Definitions

“Pollution” is defined herein as any substance(s) found in the environment (e.g., in the air, water, or food) that produces harm (e.g., discomfort, a disease state, or a decreased life span) in a multicellular organism. The multicellular organism may be a human or a non-human animal. Pollution may be generated by humans (e.g., chemicals in the air produced by industrial processes), or pollution may be naturally-occurring (e.g., arsenic in drinking water). Exposure to pollution may occur intentionally (e.g., a human smoking cigarettes) or unintentionally (e.g., a human drinking water that contains, unbeknownst to the human, pollution).

The term “cosmeceutical,” as used herein, refers to any non-pharmaceutical preparation that is used on the external body of a human or a non-human animal. Cosmeceuticals include hair care products (e.g., shampoo or conditioner), skin care products (e.g., skin lotion or soaps), and cosmetics (e.g., lipstick, blush, or eye shadow).

The term “drug” is intended to refer to a chemical entity, whether in the solid, liquid, or gaseous phase which is capable of providing a desired therapeutic effect when administered to a subject. The term “drug” should be read to include synthetic compounds, natural products and macromolecular entities such as polypeptides, polynucleotides, or lipids and also small entities such as neurotransmitters, ligands, hormones or elemental compounds. The term “drug” is meant to refer to that compound whether it is in a crude mixture or purified and isolated.

B. Disease States

In specific embodiments, the present invention involve the diagnosis and treatment of disease states. Diseases, or “disease states” include infectious diseases (e.g., a viral, bacterial, protozoan, or fungal disease), genetic diseases, diabetes, neurodegenerative diseases, and cancer. Disease states may affect any human or non-human animal.

1. Fungal Diseases

The methods of the present invention may be used to diagnose, predict responses to, and monitor responses to fungal diseases; additionally, the methods of the present invention may also be used to discover pharmaceuticals or cosmeceuticals for the treatment of fungal diseases. Fungal diseases are caused by fungal and other mycotic pathogens (some of which are described in Human Mycoses, E. S. Beneke, Upjohn Co.: Kalamazoo, Mich., 1979; Opportunistic Mycoses of Man and Other Animals, J. M. B. Smith, CAB International: Wallingford, UK, 1989; and Scrip's Antifungal Report, by PJB Publications Ltd, 1992); fungal diseases range from mycoses involving skin, hair, or mucous membranes, such as, but not limited to, Aspergillosis, Black piedra, Candidiasis, Chromomycosis, Cryptococcosis, Onychomycosis, or Otitis extema (otomycosis), Phaeohyphomycosis, Phycomycosis, Pityriasis versicolor, ringworm, Tinea barbae, Tinea capitis, Tinea corporis, Tinea cruris, Tinea favosa, Tinea imbricata, Tinea manuum, Tinea nigra (palmaris), Tinea pedis, Tinea unguium, Torulopsosis, Trichomycosis axillaris, White piedra, and their synonyms, to severe systemic or opportunistic infections, such as, but not limited to, Actinomycosis, Aspergillosis, Candidiasis, Chromomycosis, Coccidioidomycosis, Cryptococcosis, Entomophthoramycosis, Geotrichosis, Histoplasmosis, Mucormycosis, Mycetoma, Nocardiosis, North American Blastomycosis, Paracoccidioidomycosis, Phaeohyphomycosis, Phycomycosis, pneumocystic pneumonia, Pythiosis, Sporotrichosis, and Torulopsosis, and their synonyms, some of which may be fatal.

Known fungal and mycotic pathogens include, but are not limited to, Absidia spp., Actinomadura madurae, Actinomyces spp., Allescheria boydii, Alternaria spp., Anthopsis deltoidea, Apophysomyces elegans, Arnium leoporinum, Aspergillus spp., Aureobasidium pullulans, Basidiobolus ranarum, Bipolaris spp., Blastomyces dermatitidis, Candida spp., Cephalosporium spp., Chaetoconidium spp., Chaetomium spp., Cladosporium spp., Coccidioides immitis, Conidiobolus spp., Corynebacterium tenuis, Cryptococcus spp., Cunninghamella bertholletiae, Curvularia spp., Dactylaria spp., Epidermophyton spp., Epidermophyton floccosum, Exserophilum spp., Exophiala spp., Fonsecaea spp., Fusarium spp., Geotrichum spp., Helminthosporium spp., Histoplasma spp., Lecythophora spp., Madurella spp., Malassezia furfur, Microsporum spp., Mucor spp., Mycocentrospora acerina, Nocardia spp., Paracoccidioides brasiliensis, Penicillium spp., Phaeosclera dematioides, Phaeoannellomyces spp., Phialemonium obovatum, Phialophora spp., Phoma spp., Piedraia hortai, Pneumocystis carinii, Pythium insidiosum, Rhinocladiella aquaspersa, Rhizomucor pusillus, Rhizopus spp., Saksenaea vasiformis, Sarcinomyces phaeomuriformis, Sporothrix schenckii, Syncephalastrum racemosum, Taeniolella boppii, Torulopsosis spp., Trichophyton spp., Trichosporon spp., Ulocladium chartarum, Wangiella dermatitidis, Xylohypha spp., Zygomyetes spp. and their synonyms. Other fungi that have pathogenic potential include, but are not limited to, Thermomucor indicae-seudaticae, Radiomyces spp., and other species of known pathogenic genera. These fungal organisms are ubiquitous in air, soil, food, decaying food, etc. Histoplasmoses, Blastomyces, and Coccidioides, for example, cause lower respiratory infections. Trichophyton rubrum causes difficult to eradicate nail infections. In some of the patients suffering with these diseases, the infection can become systemic causing fungal septicemia, or brain/meningal infection, leading to seizures and even death.

2. Viral Diseases

The methods of the present invention may be used to diagnose, predict responses to, and monitor responses to viral diseases; additionally, the methods of the present invention may also be used to discover pharmaceuticals or cosmeceuticals for the treatment of viral diseases. Viral diseases include, but are not limited to: influenza A, B and C, parainfluenza (including types 1, 2, 3, and 4), paramyxoviruses, Newcastle disease virus, measles, mumps, adenoviruses, adenoassociated viruses, parvoviruses, Epstein-Barr virus, rhinoviruses, coxsackieviruses, echoviruses, reoviruses, rhabdoviruses, lymphocytic choriomeningitis, coronavirus, polioviruses, herpes simplex, human immunodeficiency viruses, cytomegaloviruses, papillomaviruses, virus B, varicella-zoster, poxviruses, rubella, rabies, picomaviruses, rotavirus, Kaposi associated herpes virus, herpes viruses type 1 and 2, hepatitis (including types A, B, and C), and respiratory syncytial virus (including types A and B).

3. Bacterial Diseases

The methods of the present invention may be used to diagnose, predict responses to, and monitor responses to bacterial diseases; additionally, the methods of the present invention may also be used to discover pharmaceuticals or cosmeceuticals for the treatment of bacterial diseases. Bacterial diseases include, but are not limited to, infection by the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also be useful against gram negative bacteria such as Klebsiella pneumoniae, Escherichia coli, Proteus, Serratia species, Acinetobacter, Yersinia pestis, Francisella tularensis, Enterobacter species, Bacteriodes and Legionella species and the like.

4. Protozoan Diseases

The methods of the present invention may be used to diagnose, predict responses to, and monitor responses to protozoan diseases; additionally, the methods of the present invention may also be used to discover pharmaceuticals or cosmeceuticals for the treatment of protozoan diseases. Protozoan or macroscopic diseases include infection by organisms such as Cryptosporidium, Isospora belli, Toxoplasma gondii, Trichomonas vaginalis, Cyclospora species, for example, and for Chlamydia trachomatis and other Chlamydia infections such as Chlamydia psittaci, or Chlamydia pneumoniae, for example.

5. Cancer

Certain embodiments of the present invention may be directed towards diagnosing cancer, predicting responses to certain treatments for cancer, and monitoring responses to treatments of cancer. Normal tissue homeostasis is a highly regulated process of cell proliferation and cell death. An imbalance of either cell proliferation or cell death can develop into a cancerous state (Solyanik et al., 1995; Stokke et al., 1997; Mumby and Walter, 1991; Natoli et al., 1998; Magi-Galluzzi et al., 1998). For example, cervical, kidney, lung, pancreatic, colorectal and brain cancer are just a few examples of the many cancers that can result (Erlandsson, 1998; Kolmel, 1998; Mangray and King, 1998; Mougin et al., 1998). In fact, the occurrence of cancer is so high that over 500,000 deaths per year are attributed to cancer in the United States alone.

Changes in gene expression are associated with many, if not most, forms of cancer. The maintenance of cell proliferation and cell death is at least partially regulated by proto-oncogenes. A proto-oncogene can encode proteins that induce cellular proliferation (e.g., sis, erbB, src, ras and myc), proteins that inhibit cellular proliferation (e.g., Rb, p16, p19, p21, p53, NF1 and WT1) or proteins that regulate programmed cell death (e.g., bc1-2) (Ochi et al., 1998; Johnson and Hamdy, 1998; Liebermann et al., 1998). However, genetic rearrangements or mutations to these proto-oncogenes, results in the conversion of a proto-oncogene into a potent cancer causing oncogene. Often, a single point mutation is enough to transform a proto-oncogene into an oncogene. For example, a point mutation in the p53 tumor suppressor protein results in the complete loss of wild-type p53 function (Vogelstein and Kinzler, 1992; Fulchi et al., 1998) and acquisition of “dominant” tumor promoting function. In certain embodiments of the present invention, gene expression from hair follicles could be used to observe changes in gene expression that correlate with certain kinds of cancer or changes that correlate with pre-cancerous states.

Cancer cells that may be identified by or correlate with changes in gene expression in hair follicle cells as measured using methods of the present invention include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

6. Diabetes

Diabetes is a significant and growing concern in the Western and developing world (Moran and Phillip, 2003). Diabetes includes type 1 and type 2 diabetes mellitus. Although no specific gene has yet been shown to cause diabetes, the application of the technique of microarrays will likely yield significant insights into expression patterns associated with this disease (Sparre et al., 2003). Because defective apoptosis may underlie diabetes (Kuhtreiber et al., 2003), microarray analysis can be used to identify changes in expression of genes important for apoptosis associated with diabetes. In one embodiment of the present invention, RNA expression in hair follicles could be used to predict risk to diabetes or to monitor changes in gene expression in hair follicles during treatment of diabetes.

7. Alopecia

Alopecia is a common disorder throughout the world. Hair loss can occur in many forms, including but not limited to: male- and female-pattern baldness, baldness, thinning hair, hair loss due to exposure to pharmaceuticals, chemotherapeutics, and environmental stimuli, or to inherited genes or traits. Because underexpression of fibroblast growth factor 19 (FGF-19) may underlie alopecia, microarray analysis can be used to identify changes in expression of genes important for follicular hair growth. In one embodiment of the present invention, RNA expression of FGF-19 in hair follicles can be used to predict, treat, or monitor therapeutic response to baldness or to develop novel therapeutics, pharmaceuticals, cosmeceuticals, or consumer products directed to the treatment of alopecia, baldness, or other associated forms of hair loss.

The inventors have identified fibroblast growth factor-19 (FGF-19) as a gene which is upregulated in hair follicles which may underlie baldness; specifically, the expression of FGF-19 was observed to be lower in the skin of bald subjects as compared to non-bald subjects. FGF-19 is a member of the fibroblast growth factor family of proteins and is distantly related to other members of the FGF family of cytokines. Most of the FGF family members regulate cell proliferation, migration, and differentiation during development in response to injury.

The 22 members of the FGF family have been implicated in cell proliferation, differentiation, survival, and migration. They are required for both development and maintenance of vertebrates, demonstrating high affinities for both protein and proteoglycan receptors. FGF-19, one of the most divergent human FGFs, is unique in binding solely to one receptor, FGFR4. A model for the complex of FGF-19 and FGFR4 demonstrates that unique sequences in both FGF-19 and FGFR4 are key to the formation of the ligand-receptor complex. (Harmer N J et al., 2004).

FGF-19 mRNA has been shown to be expressed in several tissues including fetal cartilage, skin, and retina, as well as adult gall bladder. The FGF-19 gene maps to chromosome 11 q13.1. FGF-19 is a high-affinity, heparin-dependent ligand for FGFR4 and exclusively binds to this receptor. (Nicholes K et al., 2002).

The inventors observed significantly increased expression of FGF-19 in the follicles of individuals with hair. FGF-19 gene expression was not present in the skin (not including follicles) taken from the head in these same individuals with hair. Similarly, expression of FGF-19 was not observed in the balding skin on the head of bald individuals.

The level of FGF-19 expression is significant in human hair follicles. FGF-19 was among the top-listed genes overexpressed in the hair follicle. Most of the other genes expressed at this or a similar level were keratin or keratin-related genes. The high level of FGF-19 gene expression in hair follicles is likely due to the fact that human hair grows continuously. Thus, FGF-19 is likely to be a critical regulator of hair growth in humans.

C. Nucleic Acid Detection and Gene Expression Analysis

RNA purified from hair follicles according to the instant invention have a variety of uses. For example, in certain embodiments, they have utility for genotyping (e.g., for identifying polymorphisms or mutations in a relevant gene) and this RNA is also critical for evaluating the degree of or absence of gene expression as well as changes in gene expression. As is well known in the art, mRNA sequences are critical for evaluating gene expression. Multiple techniques are well known in the art regarding the analysis of gene expression. These techniques include microarray analysis, differential display, Northern blots, and Southern blots.

1. Hybridization

Hybridization is a technique well known in the art that is often used in experiments concerning nucleic acids. The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences involved with the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl2, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl2, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Amplification of nucleic acids is another technique that may be used with certain embodiments of the present invention. Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to specific genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCRTM and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Of particular interest to the present invention is the use of reverse transcription (RT), reverse transcription PCR (RT-PCR), and qualitative reverse transcription PCR (Q-RT-PCR). As is well known in the art, RNA can be reverse transcribed to DNA (e.g., cDNA) via a reverse transcriptase. Many products are commercially available for performing reverse transcription. RT-PCR is well known in the art and is often used to amplify cDNA sequences. In some instances, these sequences are specific to a single gene; however, for the purposes of microarray analysis, typically multiple primers are used to insure that essentially all cDNA species are amplified. The fluorescence-based Q-RT-PCR, also known as “real-time reverse transcription PCR” is widely used for the quantification of mRNA levels and is a critical tool for basic research, molecular medicine and biotechnology. Q-RT-PCR assays are easy to perform, capable of high throughput, and can combine high sensitivity with reliable specificity (Bustin, 2002).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In other embodiments, detection is by Northern blotting and hybridization with a labeled probe. Northern blotting provides a way to measure mRNA. The techniques involved in Northern blotting are well known to those of skill in the art (Trayhurn, 1996). A cDNA labelled with ³²P is the most commonly used probe, although other methods (including non-radioactive detection methods) also exist.

In other embodiments of the invention, RNA isolated from hair follicles may be analyzed using mass spectroscopy. Since its inception and commercial availability, the versatility of matrix assisted laser desorbtion ionization time-of-flight mass spectrometry (MALDI-TOF-MS) has been demonstrated convincingly by its extensive use for qualitative analysis. MALDI-TOF-MS has been employed for both applications relating to proteins (e.g., the characterization of synthetic polymers, peptides, recombinant proteins, and protein analysis) as well as for DNA and oligonucleotide sequencing (Miketova et al., 1997; Faulstich et al, 1997; Bentzley et al., 1996).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In particular, the inventors anticipate that MALDI-TOF-MS may be used to observe expression of RNA isolated from hair samples in order to identify genes that differentially expressed due to factors including, but not limited to, the presence of a disease state and genes relating to hair development. Also, in another embodiment of the present invention, RNA from hair follicles could be reverse transcribed to cDNA, and this cDNA could be subsequently analyzed by matrix-assisted laser desorption/ionization (MALDI) techniques such as MALDI-TOF-MS.

MALDI-TOF-MS has been used for many applications, and many factors are important for achieving optimal experimental results (Xu et al., 2003). Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al, 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result. With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

Several different MALDI approaches may be used in certain embodiments of the present invention. For example, certain MALDI techniques may be used to determine specific nucleotide polymorphisms and/or for genotyping (Blondal et al., 2003; Marvin et al., 2003; Pusch et al., 2003; Tost et al., 2002; Sauer et al., 2002). In particular, these techniques may be employed in an embodiment of the present invention by genotyping and/or detecting polymorphisms in RNA obtained from hair follicles.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays for Nucleic Acid Detection

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substititution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

5. Differential Display

One embodiment of the present invention involves the use of differential display. Differential display allows a method for detecting mRNA and evaluating gene expression. Techniques involving differential display are well known in the art (Stein and Liang, 2002; Liang, 2002; Broude, 2002).

6. DNA Chips and MicroArrays

A preferred embodiment of the present invention is to use RNA from hair follicles to evaluate gene expression. One method of evaluating gene expression is DNA chips and microarrays. DNA arrays and gene chip technology provides a means of rapidly screening a large number of DNA samples for their ability to hybridize to a variety of single stranded DNA probes immobilized on a solid substrate. Specifically contemplated are chip-based DNA technologies such as those described by Hacia et al. (1996) and Shoemaker et al. (1996). These techniques involve quantitative methods for analyzing large numbers of genes rapidly and accurately. The technology capitalizes on the complementary binding properties of single stranded DNA to screen DNA samples by hybridization. Pease et al. (1994); Fodor et al. (1991). Basically, a DNA array or gene chip consists of a solid substrate upon which an array of single stranded DNA molecules have been attached. For screening, the chip or array is contacted with a single stranded DNA sample which is allowed to hybridize under stringent conditions. The chip or array is then scanned to determine which probes have hybridized. In a particular embodiment of the instant invention, a gene chip or DNA array would comprise probes specific for chromosomal changes evidencing the development of a neoplastic or preneoplastic phenotype. In the context of this embodiment, such probes could include synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change.

A variety of gene chip or DNA array formats are described in the art, for example U.S. Pat. Nos. 5,861,242 and 5,578,832 which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

In particular embodiments, a target nucleic acid may be tagged or labeled with a substance that emits a detectable signal, for example, luminescence. The target nucleic acid may be immobilized onto the integrated microchip that also supports a phototransducer and related detection circuitry. Alternatively, a gene probe may be immobilized onto a membrane or filter which is then attached to the microchip or to the detector surface itself. In a further embodiment, the immobilized probe may be tagged or labeled with a substance that emits a detectable or altered signal when combined with the target nucleic acid. The tagged or labeled species may be fluorescent, phosphorescent, or otherwise luminescent, or it may emit Raman energy or it may absorb energy. When the probes selectively bind to a targeted species, a signal is generated that is detected by the chip. The signal may then be processed in several ways, depending on the nature of the signal.

The DNA probes may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, the probes are stabilized and therefore may be used repeatedly. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules.

Binding of the probe to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nitrocellous membranes, the DNA probes are spotted onto the membranes. A UV light source (Stratalinker,™ Stratagene, La Jolla, Calif.) is used to irradiate DNA spots and induce cross-linking. An alternative method for cross-linking involves baking the spotted membranes at 80° C. for two hours in vacuum.

Specific DNA probes may first be immobilized onto a membrane and then attached to a membrane in contact with a transducer detection surface. This method avoids binding the probe onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

Laser controlled microdissection can also be used in conjunction with microarrays (Hergenhahn et al., 2003). In some embodiments, specific cells comprising or near hair follicle cells could me dissected using microdissection, and the subsequently isolated RNA could be analyzed using microarrays. In certain embodiments of the present invention, this approach may provide advantages over other approaches due to the ability to isolate specific cell types near or including hair follicle cells from plucked hairs.

D. Hair Follicles

Plucked hair provides a readily acquired “minimally” invasive source of tissue that is readily influenced by stimuli such as disease states and therapeutic agents. Specifically, plucked hair includes tissue from the hair follicle germinative region. Genes expressed in hair follicles are controlled by hereditary factors and can be modified in response to environmental changes. Hair follicles contain cells that are actively involved in the growth of the hair shaft and influence the external phenotypic characteristics of the hair shaft. The genes expressed in these cells are all expected to control the diameter of the hair shaft, the thickness of the cuticle, cortex and medulla, the amount of curl, the strength of the shaft (i.e., the amount of cross-linkage in structural proteins), the hair color, and the amount and type of lipid coating. Furthermore, the follicle is among the only organs of the body that is continuously undergoing cycles of death and regeneration. Changes in both environmental stimuli and physiology can modulate this developmental cycle and cause detectable changes in gene expression in this tissue. Until now, methods for performing gene expression analyses using RNA isolated from hair follicles have not yet been developed.

E. Screening Assays

The present invention has many applications for use in screening assays. For example, the present invention could be used to evaluate changes in expression produced by a drug. In this application, RNA from hair follicles could be obtained and analyzed from a control subject and a subject that has been exposed to a drug. Differences in gene expression could be used to determine if the drug has commercial value. For example, if a drug results in the up-regulation of expression of genes associated with apoptosis, then the drug may have value for treating cancer. In another embodiment, the drug could be replaced with a toxin (e.g., a compound occurring in the environment that produces an adverse effect for animals or plants) to provide information relating to specific changes in gene expression produced by a toxin. Information about changes in expression caused by a toxin could be used to produce new therapies for treating exposure to the toxin.

In another application, the present invention could be used to evaluate genes that are differentially expressed as a result of a disease or phenotype. For example, RNA from hair follicles could be obtained from a control subject and a subject that has a specific disease. Differences in gene expression between these subjects could be used to determine what genes are relevant for the treatment of the disease and/or affected by the disease. This information (i.e., which genes are relevant to and/or altered by a specific disease) could be used to produce new therapies for the disease. In another embodiment, RNA from hair follicles could be obtained from a control subject and a subject that has a specific phenotype (e.g., baldness). Differences in gene expression could be used to determine what genes cause and/or are affected by the phenotype. It is also anticipated that this information (e.g., genes responsible for causing baldness) could be used to produce pharmaceuticals or cosmeceuticals for altering and/or reversing the phenotype (e.g., the phenotype of baldness). In all of the above examples, the exact number of subjects may be altered to produce an appropriate level of statistical power, and in most embodiments multiple subjects will be included in both the control group of subjects and the treated (i.e., exposed to a drug, disease, or toxin, or displaying a particular phenotype) group of subjects.

The present invention further comprises methods for identifying modulators of the expression of FGF-19. These assays may comprise random screening of large libraries of candidate substances; alternatively, the assays may be used to focus on particular classes of compounds selected with an eye towards structural attributes that are believed to make them more likely to modulate the function of FGF-19.

To identify an FGF-19 modulator, one generally will determine the expression of FGF-19 in the presence and absence of the candidate substance, a modulator defined as any substance that alters function. For example, a method generally comprises:
(a) providing a candidate modulator;
(b) admixing the candidate modulator with an isolated compound or cell, or a suitable experimental animal;
(c) measuring one or more characteristics of the compound, cell or animal in step (c); and
(d) comparing the characteristic measured in step (c) with the characteristic of the compound, cell or animal in the absence of said candidate modulator,

wherein a difference between the measured characteristics indicates that said candidate modulator is, indeed, a modulator of the compound, cell or animal.

Assays may be conducted in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially inhibit or enhance expression of a target. The candidate substance may be a protein or fragment thereof, a small molecule, or even a nucleic acid molecule. In certain preferred embodiments of the present invention, useful pharmacological compounds that are structurally related to fibroblast growth factors may be identified and used for the treatment of baldness. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on gene expression in epithelial and/or follicular cells (e.g., causing decreases in expression of FGF-19). Regardless of the type of inhibitor or activator identified by the present screening methods, the effect of the inhibition or activator by such a compound results in alteration of gene expression in epithelial and/or follicular cells (e.g., downregulation of FGF-19) as compared to that observed in the absence of the added candidate substance.

2. In Cyto Assays

The present invention also contemplates the screening of compounds for their ability to modulate FGF-19 in cells. Various cell lines can be utilized for such screening assays, including cells specifically engineered for this purpose. Human epithelial cell lines, skin cell lines, human gall bladder cell lines, or multiple human fetal tissue cell lines may be used to detect quantative levels of FGF-19 expression and determine up- or down-regulation of expression of FGF-19.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

3. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that upregulates FGF-19 expression In these embodiments, the present invention is directed to a method for determining the ability of a candidate substance to induce gene expression of FGF-19 in human epithelial or follicular cells, generally including the steps of: administering a candidate substance to the animal; and determining the ability of the candidate substance to reduce one or more characteristics of FGF-19 regulation. FGF-19 is not endogenous in some animal species and is highly divergent in humans and transgenic animals.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Isolation of RNA from Hair Follicles

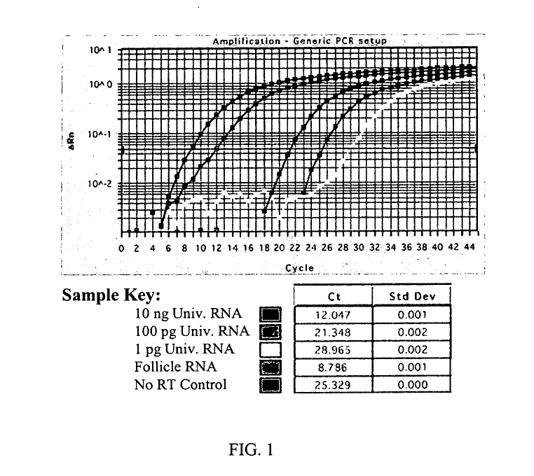

The inventors have determined that RNA from hair follicles can be used to perform gene expression analysis, and have also established a method for the isolation of RNA from hair follicles. In this method, hair is plucked from the scalp with tweezers, and total RNA is prepared immediately on collected samples. In one experiment, 10 follicles were collected from a volunteer's scalp. The hair was trimmed and RNA was prepared using the Qiagen RNeasy Mini kit. The presence of a transcript was assessed by performing quantitative PCR on the 18S ribosomal housekeeping gene (FIG. 1).

Surprisingly, as shown by detection of the 18S ribosomal housekeeping gene in FIG. 1, genes can be detected using RNA from hair follicles. Amounts of material were assessed by quantitating yields of housekeeping gene in this material to a commercially available preparation of RNA (Universal RNA control, Stratagene). The approximate signal/follicle was equal to 100 ng of input control RNA, suggesting yields are adequate for differential gene expression studies.

However, the problem of RNA degradation was also observed. These studies were performed using pulled hairs from several distinct donors. Significant variance in the integrity from different individuals was observed. A quality-control check of pulled hair total RNA was performed using agarose gel electrophoresis. Samples were prepared and analyzed as described above. Total RNA from peripheral blood mononuclear cells (control RNA of high quality, exhibiting low levels of degradation) was compared to total RNA preparations from 4 individuals. In non-degraded RNA preparations, the ratio of 28S to 18S bands is close to 1.8. This ratio is closer to 1 for the total RNA obtained from hair follicles from several of the individuals. Additionally, spectrophotometric readings can overestimate RNA quantity. Thus, RNA degradation can occur and presents a problem that must be overcome in order to use RNA from hair follicles for gene expression analysis.

Although hair follicles can provide sufficient amounts of RNA for microarray analysis, RNA degradation can occur when RNA is isolated and subsequently shipped to another location for analysis. For example, RNA was purified from Philpott hair samples in Germany using either Trizol or a combination of Trizol plus RNeasy (the latter termed “NIH” protocol). Yields from ten follicles were 3.06 μg and 0.45 μg with the Trizol and NIH protocol, respectively. 2.0 μg of total RNA is the standard amount used for our microarray experiments. RNA quality was assessed in the U.S. using an Agilent 2100 Bioanalyzer capillary gel electrophoresis system (Agilent Technologies, Waldbronn, Germany). Electropherograms of RNA obtained from Germany demonstrated partial degradation of RNA. Intact (non-degraded) total RNA typically has a ratio of 28s:18s rRNA (obtained from the area under the curve) of 2.0. For microarray purposes, ratios as low as 1.5 are often considered acceptable. The RNA ratios of Philpott samples obtained from Germany were significantly below 1.0, demonstrating that significant degradation of RNA had occurred. When fluorescently-labeled cDNA produced from this partially degraded RNA was labeled and hybridized to microarrays, numerous genes, including those encoding particular hair keratins, were detected as expressed above background (Table 1). While this suggested that tissue-specific or tissue-related gene expression was observed, the low level of expression of nearly all genes (maximum signal intensity is 65,536), as well as the quality of the RNA, led to obvious concerns of how well such microarray data reflect transcripts in cells of hair follicles.

TABLE 1
Low Signal Intensity Microarray Expression
of Hair Keratin RNA from Germany

Hair keratin type (signal intensity)

| | Acidic | Basic |
|---|---|---|
| Expressed: | 4 (700) | 6 (2500) |
| | 1, 8, and 6 (500) | 3 (1300) |
| | 2 (300) | 2 (1300) |
| Not expressed: | 3B, 5, 7 | 1, 4 |

For these reasons, a series of experiments was begun here to optimize the quality of RNA from hair tissue. For this purpose, hair follicles were pulled and RNA was isolated using various methods. As measured using RNA isolated from the hair follicles of several individuals, the quality of the RNA using the NIH protocol varied greatly between samples but consistently showed RNA degradation. Other methods tested included Trizol alone, snap-freezing samples in liquid nitrogen followed by Trizol or NIH purification procedures, Trizol followed by RNeasy, and RNeasy alone. Use of the latter product consistently yielded high quality, intact RNA from hair follicles. The presence of 28s rRNA in these samples, the high 28s: 18s rRNA ratio, and the elimination of low molecular weight RNA species was consistently observed in electropherograms using this approach.

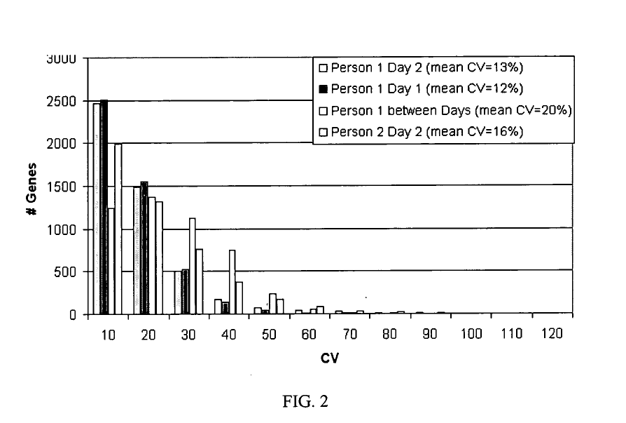

These different methods of hair follicle RNA isolation were compared using microarray gene expression analysis (further described in Example 2). Data were collected from multiple microarrays hybridized with hair follicular RNA from different individuals after purification with either RNeasy or the NIH protocol. The number of genes dramatically increased when RNeasy was used, in some cases by an order of magnitude. Furthermore, the RNeasy-purified RNA samples produced highly reproducible results. Using independently isolated samples of RNA from two different individuals, the distribution of the coefficients of variation (CV) for all genes was narrow, and the within individual mean CV was 12 and 16%, respectively (FIG. 2). The distribution and mean CV was nearly identical for independent samples isolated from the same person on different days (mean CV=12-13%); however, the distribution and mean CV increased when samples from different days were compared (mean CV=20%). Comparison of microarray data from Philpott/NIH protocol to the optimized pulled hair follicle/RNeasy protocol revealed similar patterns of hair keratin gene expression, but the latter results produced between 3- to 18-fold more intense signals than the Philpott/NIH samples. Furthermore, the number of expressed genes increased dramatically (Table 2) to levels typically detected with other types of tissue. Based on these findings, the inventors believe that microarray data from RNA isolated with RNeasy will provide highly reproducible results that accurately reflect the pattern of expression of RNA in hair follicles for this research project.

TABLE 2
Microarray Data: Greater # of Genes Detected and Reduced
Variation in # of Genes Using RNeasy Method

| | Range of # |
|---|---|
| | genes expressed |
| Philpott RNA/NIH Method | |
| Individual 1, different microarrays | 720-2232 |
| Individual 2, different microarrays | 300-6088 |
| Variation between other individuals | 987-7105 |
| Pulled Hair Follicle RNA/Rneasy Method | |
| Individual 1, different microarrays | 8526-10,313 |
| Individual 2, different microarrays | 8468-10,140 |
| Different individuals | 8524-9191 |

Ranges represent 95% confidence intervals.

### Example 2

### Gene Analysis of RNA from Hair Follicles Using Microarrays

In collaboration with Qiagen Operon, next-generation genome-scale oligo-based human and mouse arrays were developed. The arrays were produced using commercially available libraries of 70 base pair long DNA oligos such that length and sequence specificity are optimized, reducing or eliminating the cross-hybridization problems encountered with cDNA-based arrays. The human arrays used here have 21,329 human genes represented.

Oligo probes on these arrays were derived from the UniGene and RefSeq databases (www.ncbi.nlm.nih.gov). The RefSeq database represents an effort by the NCBI to create a true reference database of genomic information for all genes of known function. For the genes present in this database, information on gene function, chromosomal location, and reference naming are available; this allows one to perform functional analyses, to identify candidate genes in disease-gene investigations, and to compare data from mouse models and humans directly from identification of differentially expressed genes which we find to be specific for hair follicles. A complete listing of the genes can be seen at the following web address: www.operon.com/arrays/humangenome.prn.

There are approximately 11,000 genes of known function in the human genome; all of these genes are represented on the array used here. Identification of biologic pathways relevant to hair development can therefore be done in a comprehensive manner. Moreover, most genes of undefined function (10,000) are also included here to facilitate novel gene discovery. Using custom bioinformatics tools (described below) these two elements have synergized in studies of human disease such that undiscovered foundations of pathophysiology and novel elements within these pathways have been uncovered. These elements should provide the same predictive potential for ongoing studies.

Specific bioinformatics approaches were used to analyze the data produced from microarray analyses. Data normalization and identification of differentially expressed genes was performed in the bioinformatics section of the OMRF Microarray Research Facility using biostatistics software developed by the OMRF microarray research facility bioinformaticians. The approach used here, denoted the “Associative Method,” associates variation in experimental gene expression to a common standard derived from a family of low variability genes derived from control experiments. The Associative Method enhances the sensitivity of analysis greater than previous modifications of the T-test and increases the number of differentially expressed genes identified without significantly increasing the misidentification of false positives. Recently a novel clustering procedure was developed at OMRF based on the calculation of positive and negative correlations among groups of hypervariable genes. This method prioritizes cluster definitions using a simple yet powerful parameter—cluster size. The Associative Method provides a means for identifying the most prominent aspects of co-regulation in a given system as well as the disregulation caused by the experimental conditions under study. The method identifies biologic pathways underlying the experimental conditions assayed. As shown in Example 3, when applied to data obtained from microarrays using peripheral blood leukocytes from juvenile rheumatoid arthritis patients, it successfully identified clinically relevant pathways underlying the pathophysiology of disease.

This microarray approach, using RNA from hair follicles, has many applications. Recent advances in the field of genomics, which include the completion of a draft sequence of the human genome, have been dramatic. Gene expression profiling on hair samples using the human oligo arrays and utilizing the cutting edge bioinformatics approaches described here are useful for gene discovery. This approach will complement the gene discovery analysis that is planned.

When analyzing gene expression, false positives can be avoided. Increased statistical power can be obtained by testing larger numbers of individuals, increasing the ability to discern genes relevant to specific phenotypes (e.g., hair development). This results from the fact that some genes exhibit natural biologic variation in expression levels. If small numbers of individuals are studied some of these genes may appear to be differentially expressed in a given tissue by chance. When tens of thousands of genes are being studied simultaneously the influence of these false positives on the results can be substantial. By studying larger numbers of individuals, biologic variation can be accurately assessed and its influence on gene identification can be minimized. Additionally, these analyses can be performed on ethnically diverse individuals such that genes contributing to a given phenotype (e.g., hair color) can be identified. The methods described here (i.e., collecting follicles by pulling hair) provides a non-invasive way to easily obtain tissue samples from a large number of individuals.

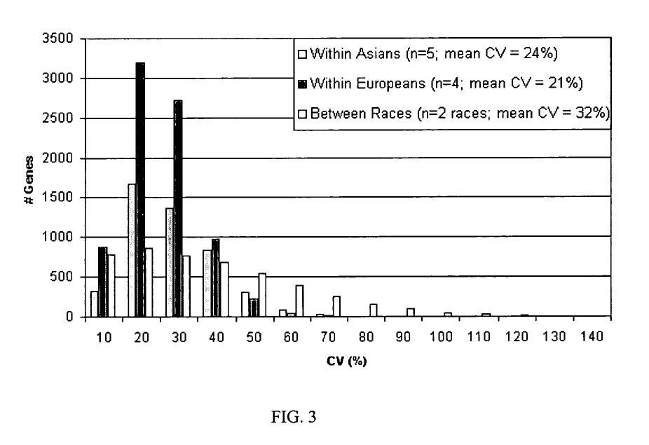

Using the optimized method of RNA purification described in Example 1, the ability of microarrays to distinguish different patterns of gene expression in hair follicles obtained from different individuals was tested. RNA was isolated from pulled hair follicles from five healthy Asian-Americans and from 4 healthy Europeans temporarily living in the U.S. The average amount of RNA was 7.5 ug per person (range=2.1-12.6 μg; 2 μg are needed for microarrays) with an average A₂₆₀:A₂₈₀ ratio of 2.05 (range=2.03-2.11). Coefficients of variation for gene expression of each group are shown in FIG. 3. As expected, the mean CV is greater for a group of individuals than for samples obtained from the same individual at different times. However, the mean CV within each race was low, and the distribution of CVs was narrow. The mean and distribution of CVs between different races was higher than that detected within each race. These increased CVs may reflect biological differences in hair follicle gene expression between races. Samples from additional people that differ in sex and ethnic origin (Asian, European and African) can be collected for a detailed analyses of differential gene expression in normal hair samples.

### Example 3

### Identification of Genes of Interest from Analysis of RNA from Hair Follicles

Genes of interest were identified using microarray gene expression analysis on hair follicle RNA obtained from healthy individuals. Using the OMRF 21,000+human gene microarray (described in Example 2), 12 genes whose expression significantly differed between Asians and Europeans were identified. Among them were genes that are known to affect hair physiology. For example, the vitamin D receptor transcript showed decreased expressed in Europeans. Point mutations in this gene reportedly produce generalized atrichia with papular lesions resulting in a failure of the first postnatal hair growth cycle. The ectodysplasin receptor was expressed in all Europeans but expressed in only one Asian. Point mutations in this gene result in a sparse hair disorder (autosomal hypohidrotic ectodermal dysplasia).

Next, the hypothesis that disease-related changes in gene expression can be identified by gene expression analysis of hair follicle RNA was evaluated. In these experiments, RNA from pulled hair follicles was isolated from 3 patients with rheumatoid arthritis (RA) and from 4 healthy individuals. All samples were from Caucasians with brown hair. RNA was isolated and cDNA was fluorescently labeled and hybridized to microarrays. Slightly more variation in gene expression was detected in the RA samples (mean CV=27%) than the control samples (mean CV=21%). It is possible that this increased variation may reflect heterogeneity in the disease process and/or treatment of these patients. On a gene-specific basis, a number of very interesting findings were obtained. 130 genes were uniquely expressed in RA patients, including transcripts for the type II bone morphogenic protein receptor, daxx which encodes a protein that binds Fas and mediates apoptosis, and an IL-6 signal transducer for which point mutations in mice have been shown to produce a RA-like disease. An interleukin-3-inducible kinase transcript was over-expressed in the RA samples relative to the controls (p<0.001). This protein induces osteoclast (one of the cells required for bone repair) differentiation. Unique and differential expression was detected for a number of other genes in this study.

These findings suggest that hair tissue is a feasible source of tissue to elucidate pathophysiologic mechanisms, and that microarray results from this tissue may be applicable to the discovery and development of potential targets for drugs that alter homeostasis. Based on these observations, these results demonstrate that it is reasonable to expect that these methods using RNA from hair follicles will be used to define molecular differences relevant to disease states; additionally, this approach will be used to identify genes involved in hair phenotype and cosmetically relevant aspects of hair follicle physiology.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Blondal T, Waage B G, Smarason S V, Jonsson F, Fjalldal S B, Stefansson K, Gulcher J, Smith A V. “A novel MALDI-TOF based methodology for genotyping single nucleotide polymorphisms. Nucleic Acids Res. 2003 Dec. 15; 31(24):e155
Marvin L F, Roberts M A, Fay L B. “Matrix-assisted laser desorption/ionization time-of-flight mass spectrometry in clinical chemistry.” Clin Chim Acta. 2003 November; 337(1-2):11-21.
Pusch W, Flocco M T, Leung S M, Thiele H, Kostrzewa M. “Mass spectrometry-based clinical proteomics. Pharmacogenomics. 2003 July; 4(4):463-76. Review.
Tost J, Gut I G. “Genotyping single nucleotide polymorphisms by mass spectrometry. Mass Spectrom Rev. 2002 Nov-Dec; 21(6):388-418. Review.
Sauer S, Gut I G. “Genotyping single-nucleotide polymorphisms by matrix-assisted laser-desorption/ionization time-of-flight mass spectrometry.” J Chromatogr B Analyt Technol Biomed Life Sci. 2002 Dec. 25; 782(1-2):73-87. Review.
Bellus, 1994
European Application No. 320 308
European Application No. 329 822
Frohman, In: PCR PROTOCOLS: A GUIDE TO METHODS AND APPLICATIONS, Academic Press, N.Y., 1990.
GB Application No. 2 202 328
Innis et al., “DNA sequencing with Thermus aquaticus DNA polymerase and direct sequencing of polymerase chain reaction-amplified DNA,” Proc Natl Acad Sci U S A. 85(24):9436-9440, 1988.
Kwoh et al., “Transcription-based amplification system and detection of amplified human immunodeficiency virus type 1 with a bead-based sandwich hybridization format, Proc Natl Acad Sci USA. 86(4):1173-1177, 1989.
Ohara et al., “One-sided polymerase chain reaction: the amplification of cDNA,”
PCT Application No. PCT/US87/00880
PCT Application No. PCT/US89/01025
PCT Application WO 88/10315
PCT Application WO 89/06700
PCT Application WO 90/07641
Proc Natl Acad Sci USA. 86(15):5673-5677, 1989.
Sambrook et al., In: Molecular Cloning: A Laboratory Manual, Vol. 1, Cold Spring Harbor Laboratory Press, Cold Spring Harbor, N.Y., Ch. 7,7.19-17.29, 1989.
U.S. Pat. No. 4,683,195
U.S. Pat. No. 4,883,750
U.S. Pat. No. 4,946,773
U.S. Pat. No. 5,279,721
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,483
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,882,864
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,630
U.S. Pat. No. 5,922,574
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 4,683,202
U.S. Pat. No. 4,800,159
U.S. Pat. No. 5,849,486
U.S. Pat. No. 5,851,772
U.S. Pat. No. 5,900,481
U.S. Pat. No. 5,919,626
Walker et al., “Strand displacement amplification—an isothermal, in vitro DNA amplification technique,” Nucleic Acids Res. 20(7): 1691-1696, 1992
Trayhurn P. “Northern blotting.” Proc Nutr Soc. (1996) Mar; 55(1B):583-9. Review.
Stein J, Liang P. “Differential display technology: a general guide.” Cell Mol Life Sci. 2002 August; 59(8):1235-40. Review.
Liang P. “A decade of differential display.” Biotechniques. 2002 August; 33(2):338-44, 346. Review.
Broude N E. “Differential display in the time of microarrays.” Expert Rev Mol Diagn. 2002 May; 2(3):209-16. Review.
Schlake T, Boehm T. “Expression domains in the skin of genes affected by the nude mutation and identified by gene expression profiling. Mech Dev. 2001 December; 109(2):419-22.
Carroll J M, McElwee K J, E King L, Byrne M C, Sundberg J P. “Gene array profiling and immunomodulation studies define a cell-mediated immune response underlying the pathogenesis of alopecia greata in a mouse model and humans. J Invest Dermatol. 2002 August; 119(2):392-402.
Miketova and Schram, Mol. Biotechnol., 8(3):249-253, 1997.
Faulstich et al., Anal. Chem., 69(21):4349-4353, 1997.
Bentzley et al., Anal. Chem., 68(13):2141-2146, 1996.
Wang et al., J. Agric. Food. Chem., 47:1549, 1999.
Wang et al., J. Agric. Food. Chem., 47:2009, 1999.
Jiang et al., J. Agric. Food Chem., 48:3305, 2000.
Wang et al., J. Agric. Food. Chem., 48:2807, 2000.
Wang et al., J. Agric. Food. Chem., 48:3330, 2000.
Yang et al., J. Agric. Food. Chem., 48:3990, 2000.
Wittmann et al., Biotechnol. Bioeng., 72:642, 2001.
Muddiman et al., Fres. J. Anal. Chem., 354:103, 1996.
Nelson et al., Anal. Chem., 66:1408, 1994.
Duncan et al., Rapid Commun. Mass Spectrom., 7:1090, 1993.
Gobom et al., Anal. Chem. 72:3320, 2000.
Wu et al., Biochem. Biophys. Res. Commun., 233(1):221-6, 1997.
Mirgorodskaya et al., Rapid Commun. Mass Spectrom., 14:1226, 2000.
Bahr et al., J. Mass. Spectrom., 32:1111, 1997.
Takach et al., J. Protein Chem., 16:363, 1997.
Xu Y, Bruening M L, Watson J T. “Non-specific, on-probe cleanup methods for MALDI-MS samples.” Mass Spectrom Rev. 2003 Nov-Dec; 22(6):429-40.
Bustin S A. “Quantification of mRNA using real-time reverse transcription PCR (RT-PCR): trends and problems.” J Mol Endocrinol. 2002 August; 29(1):23-39. Review.
Moran O, Phillip M. “Leptin: obesity, diabetes and other peripheral effects—a review.” Pediatr Diabetes. 2003 June; 4(2):101-9.
Sparre T, Bergholdt R, Nerup J, Pociot F. “Application of genomics and proteomics in Type 1 diabetes pathogenesis research. Expert Rev Mol Diagn. 2003 November; 3(6):743-57.
Harmer N J, et al., The crystal structure of fibroblast growth factor (FGF) 19 reveals novel features of the FGF family and offers a structural basis for its unusual receptor activity. Biochemistry. 2004 Jan. 27; 43(3):629-40.
Nicholes K, et al. A mouse model of hepatocellular carcinoma: ectopic expression of fibroblast growth factor 19 in skeletal muscle of transgenic mice. Am J Pathology. 2002 June; 160(6):2295-307.

What is claimed is:

1. A method for determining gene expression comprising the steps of:
a) obtaining a hair follicle;
b) isolating RNA from said hair follicle; and
c) detecting said RNA by performing gene expression analysis.

2. The method of claim 1, wherein said hair follicle is obtained from a human.

3. The method of claim 2, wherein said RNA is stabilized.

4. The method of claim 3, wherein said RNA is amplified.

5. The method of claim 4, wherein said RNA is reverse transcribed into DNA.

6. The method of claim 5, wherein said gene expression analysis is microarray analysis.

7. The method of claim 6, wherein said hair follicle is obtained by plucking a hair.

8. The method of claim 1, wherein said gene expression analysis is Northern blot analysis or Southern blot analysis.

9. The method of claim 1, wherein said gene expression analysis is differential display.

10. The method of claim 1, wherein said gene expression analysis is used for disease diagnosis, for predicting outcomes for the treatment of a disease, or for monitoring a response to a stimuli.

11. The method of claim 10, wherein said disease comprises a disease selected from the group consisting of cancer, a genetic disease, a viral disease, a fungal disease, a bacterial disease, a cardiac disease, diabetes, a neurodegenerative disease, or an autoimmune disease.

12. The method of claim 10, wherein said stimuli is a pharmaceutical or a cosmeceutical.

13. The method of claim 10, wherein said stimuli is an environmental stimuli.

14. The method of claim 13, wherein said environmental stimuli is pollution or a toxin.

15. A method for evaluating the biological activity of a candidate substance comprising the steps of:
a) exposing a subject to the absence or presence of said candidate substance;
a) obtaining a hair follicle from said subject;
b) isolating RNA from said hair follicle; and
c) detecting said RNA by performing gene expression analysis,
wherein a difference in said gene expression analysis is observed as a result of said exposure to said candidate substance as compared to said absence of said exposure to said candidate substance.

16. The method of claim 15, wherein said RNA is stabilized.

17. The method of claim 16, wherein said RNA is amplified.

18. The method of claim 17, wherein said RNA is reverse transcribed into DNA.

19. The method of claim 18, wherein said gene expression analysis is microarray analysis.

20. The method of claim 19, wherein said subject is a human.

21. The method of claim 20, wherein said candidate substance can affect hair development.

22. The method of claim 19, wherein said candidate substance is used to treat a disease selected from the group consisting of: cancer, a genetic disease, a viral disease, a fungal disease, a bacterial disease, a cardiac disease, diabetes, a neurodegenerative disease, or an autoimmune disease.

23. The method of claim 15, wherein said gene expression analysis is Northern blot analysis or Southern blot analysis.

24. The method of claim 15, wherein said gene expression analysis is differential display.

25. The method of claim 15, wherein said difference comprises a difference in expression of fibroblast growth factor-19.
